(19) United States
(12) Patent Application Publication
(10) Pub. No.: US 2020/0066408 A1
(43) Pub. Date: Feb. 27, 2020

(54) SYSTEM AND METHOD FOR DETERMINING EFFECTIVE TREATMENT PLANS

(71) Applicant: SYSTEMS4PT, LLC, West Redding, CT (US)

(72) Inventors: Dan Alloway, Bethel, CT (US); Alain Momdjol, Silver Spring, MD (US)

(21) Appl. No.: 16/665,161

(22) Filed: Oct. 28, 2019

Related U.S. Application Data
(63) Continuation-in-part of application No. 14/314,174, filed on Jun. 25, 2014, now abandoned.
(60) Provisional application No. 61/839,109, filed on Jun. 25, 2013.

(51) Int. Cl. G16H 50/20; A61B 5/00; G16H 50/70

(52) U.S. Cl. CPC G16H 50/20; G16H 50/70; A61B 5/4836

(57) ABSTRACT

A treatment plan effectiveness system (TPES) allows for the receipt of information related to a patient's functional deficits and from that information provides one or more treatment plans meeting predetermined criteria. TPES can provide an outcome focused analysis of available treatment plans available for a given diagnosis and functional deficit set, with statistical support, thus allowing a medical service provider to provide better, more consistent, substantiated care.

Outcomes, after or during treatment plan administration, are added to TPES thereby enhancing the useful knowledge available to medical service providers serving future patients. A TPES according to the present disclosure can also provide automated evidence based documentation in support of a given treatment plan, for treatment plan decision making or for reimbursement.

### RELATED APPLICATION DATA

This application claims is a continuation-in-part of U.S. application Ser. No. 14/314,174, filed Jun. 25, 2014 and titled “System and Method for Determining Effective Treatment Plans,” which claims priority to U.S. Application Ser. No. 61/839,109, filed Jun. 25, 2013, entitled “Computer Implemented Methods And Systems For Determining Effective Plans Of Care And Generating Evidence-Based Documentation,” each of which is incorporated by reference in its entirety.

### FIELD OF THE INVENTION

The present invention generally relates to health care treatment systems. In particular, the present invention is directed to a System and Method for Determining Effective Treatment Plans.

### BACKGROUND

Providers of medical services have the responsibility to properly document and manage large volumes of data related to each of their patients to assess the most effective treatment methods and to obtain reimbursement for services provided. For most medical services, a patient is only responsible for paying a portion of the cost for their treatment and a third party, such as an insurance company or a governmental program, is responsible for paying the remainder of the costs. These third parties are often referred to as “payers”.

In an effort to improve patient care and reduce expenses and fraud. payers have started requiring more detailed explanations and in some cases, evidence-based documentation before they will submit payment for a given medical treatment or service. Evidence-based documentation details the treatment plan of care that will restore the patient's functional deficits, including supporting evidence that proves that these positive outcomes will likely occur. Although evidence-based documentation appears to be a reasonable request from the payers' perspective, this documentation shift has been an unwelcome and stressful requirement for medical service providers. These providers now have the burden of researching and finding evidence to support each treatment plan they propose, which takes a great deal of time away from actually treating the patient. Moreover, providers do not have access to updated data about evidence that is associated with particular treatment plans, access to new treatment plans based on functional deficits, or have the benefit of data for patients from other, disparately located medical service providers.

The related art in this area focuses on industry-wide solutions which try to make it easier for therapists and medical providers to find the scientific articles and research studies to support their treatment plans and, while that is important, these systems do not provide a practical solution to medical providers or do not provide statistical based evidence for each treatment plan available to the medical providers.

### SUMMARY OF THE DISCLOSURE

In a first exemplary aspect, a method is disclosed for developing an evidence-based treatment plan for a patient having functional deficits by executing a computer-executable instructions stored on a non-transitory computer-readable medium, the method comprising, the steps of: receiving, as inputs, a standardized outcome test (“SOT”) data representative of the patient's functional deficits and a diagnosis category; generating an SOT score based upon the SOT data; accessing a database comprising a plurality of treatment plans and a plurality of supporting documentation, wherein each of the plurality of treatment plans is associated with: an average initial SOT score; one or more output measures including an average final SOT score and an average number of patient visits; and an indicator of supporting documentation; providing a list of ones of the plurality of treatment plans that are associated with the diagnosis category and the average initial SOT score comparable to the SOT score; selecting, from the list, one of the plurality of treatment plans based upon at least one of the one or more output measures and the indicator of supporting documentation; and generating an evidence based documentation statement based upon the selecting, the evidence based documentation statement being formulated from ones of the plurality of supporting documentation associated with the selected treatment plan.

In another exemplary aspect, an electronic system is disclosed for developing an evidence-based treatment plan for a patient having functional deficits and a diagnosis category, the electronic apparatus comprising: a processor; and a non-transitory computer readable medium in communication with the processor; wherein the non-transitory computer readable medium includes instructions that cause the processor to: generate a standardized outcome test (sot) score based upon the patient's functional deficits; access a database comprising a plurality of treatment plans and a plurality of supporting documentation, wherein each of the plurality of treatment plans is associated with: an average initial sot score; one or more output measures including an average final sot score and an average number of patient visits; and an indicator of supporting documentation; and compare the sot score with each respective one of the average initial sot scores; and provide a list of ones of the plurality of treatment plans that are associated with the diagnosis category and the average initial sot score comparable to the SOT score.

In yet another exemplary aspect, an evidenced based treatment plan development system is disclosed that comprises a processor; and a non-transitory computer readable medium in communication with the processor, the non-transitory computer readable medium including: an input module for requesting and receiving an standardized outcome test (SOT) score related to a condition of a patient; a plan of treatment engine configured to: receive the SOT score; compare the SOT score to a database of treatment plans corresponding to the condition of the patent, wherein each of the treatment plans includes a SOT average, the SOT average being a reported actual result from a former patient; and prepare a list of ones of the database of treatment plans where the SOT average is comparable to the SOT score; and an evidence based documentation (EBD) engine configured to: match a supporting documentation document to each of the ones of the database of treatment plans in the list; generate an evidence based documentation statement based upon a selected one of the ones of the database of treatment plans in the list.

In another exemplary embodiment, a system for determining a likely most effective treatment plan for a patient in need thereof is provided in which the patient has identified and quantified functional deficits and a patient diagnosis category determined by a health care provider. The system includes a database including information related to a plurality of other patients, the information including at least one diagnosis category, an initial standardized outcome test (SOT) score, a final SOT score, a treatment plan, and a treatment plan duration, wherein at least one of the associated treatment plans is unknown to the health care provider or wherein the health care provider is unaware as to an efficacy of any particular treatment plan. The system also includes a computing device including a processor and a non-transitory computer readable medium in communication with the processor and the database, wherein said non-transitory computer readable medium includes instruction that cause the processor to generate a SOT score based upon the patient's functional deficits, identify a plurality of the treatment plans that address the patient diagnosis category, compare the SOT score to the initial SOT scores of the plurality of treatment plans, and select, from the plurality of identified treatment plans, the likely most effective treatment plan by evaluating the final SOT score and the treatment plan duration so as to develop a rate of improvement for each of the plurality of identified treatment plans, wherein the treatment plan with the highest rate of improvement is the likely most effective treatment plan.

In another exemplary aspect, a method for selecting a treatment plan for patients having identifiable functional deficits is provided that includes providing a database accessible to a plurality of disparately located health care providers, each of the health care providers having a plurality of patients, receiving information from the plurality of disparately located health care providers, the information including initial standardized outcome test (SOT) data, final SOT data, diagnosis category, treatment plan duration, and treatment plans, wherein each of the forgoing relating to each of the plurality of patients, evaluating the information so as to develop a rate of improvement for each diagnosis category based upon the treatment plan duration, the initial SOT data and the final SOT data, and selecting a likely most effective treatment plan based upon the treatment plan with the highest rate of improvement.

In another exemplary aspect, a method of improving patient treatment plans is provided that includes providing a database accessible to a plurality of disparately located health care providers, each of the health care providers having a plurality of patients, receiving information from the plurality of disparately located health care providers, the information including initial standardized outcome test (SOT) data, final SOT data, diagnosis category, treatment plan duration, and treatment plans, wherein each of the forgoing relating to each of the plurality of patients, evaluating the information so as to develop a rate of improvement for each diagnosis category based upon the treatment plan duration, the initial SOT data, and the final SOT data, and updating the rate of improvement based upon additional information received from the plurality of disparately located health care providers.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of a treatment plan effectiveness system (TPES) according to an embodiment of the present invention;

FIG. 2 is another block diagram related to the TPES shown in FIG. 1 according to an embodiment of the present invention;

FIG. 3 is an exemplary table of a plurality of treatment plans and related information according to an embodiment of the present invention;

FIG. 4 is an exemplary evidence-based statement and documentation statement according to an embodiment of the present invention;

FIG. 5 is a block diagram of a process of developing an evidence-based treatment plan according to an embodiment of the present invention;

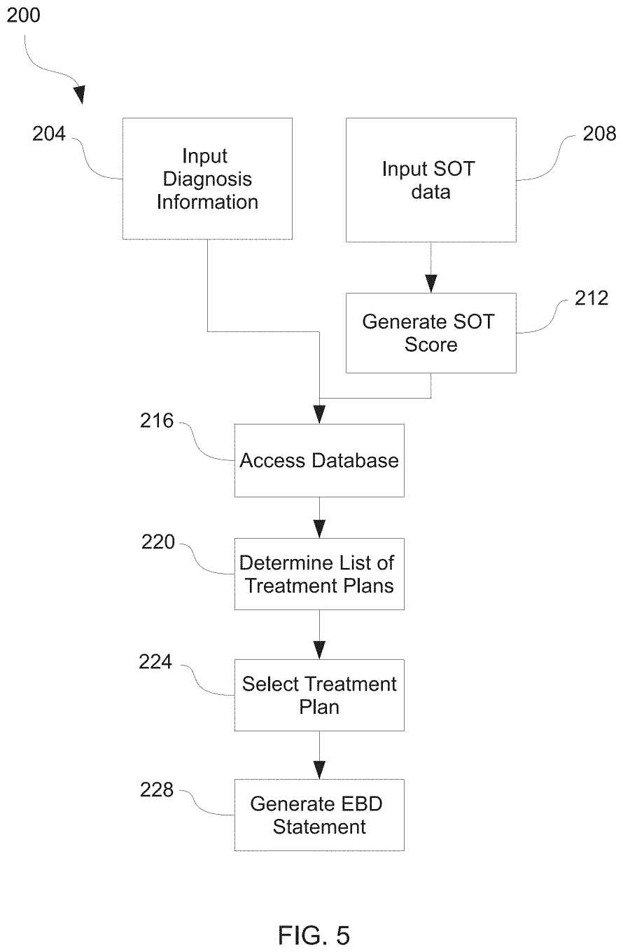

FIG. 6 is a block diagram of a process of altering a treatment plan according to an embodiment of the present invention; and

FIG. 7 is a block diagram of a computing system suitable for use with a systems and methods of determining effective treatment plans according to embodiments of the present invention.

### DESCRIPTION OF THE DISCLOSURE

The following embodiments of the invention may be implemented using hardware or software or any combination of the two where desired.

A treatment plan effectiveness system (TPES) according to the present disclosure allows for the receipt of information related to a patient's medical condition and the type and severity of the patient's functional deficits, and from that information provides one or more treatment plans meeting predetermined criteria. The TPES can provide an outcome focused analysis of available treatment plans available for a given diagnosis and functional deficit set, with statistical support, thus allowing a medical service provider an objective tool to assist in the provision of better, more consistent, substantiated care than is possible for providers practicing conventionally. Outcomes, after or during treatment plan administration, are added to TPES thereby enhancing the useful knowledge available to medical service providers serving future patients as well as to disparately located and unaffiliated medical service providers. TPES according to the present disclosure can also provide automated evidence based documentation in support of a given treatment plan, facilitating treatment plan decision making or for reimbursement. With the understanding that the universe of treatment plans is not stagnant, the TPES can also allow for medical service providers to alter existing plans to meet the needs of patients and store information related to those altered plans for use by other medical service providers. In this way; health care providers can gain the benefit of data related to patients that they do not treat or have access to otherwise as well as the accumulation of information that cannot be processed by the health care provider herself (it is worth noting, as well, that many details regarding patients cannot be transmitted between providers of medical services without the patient's permission due to health care records privacy laws). The vast amount of patient data, treatment plan data, and evidenced based documentation cannot be meaningfully processed by health care providers in a manner that allows for the selection of appropriate treatment plans based on statistical, objective evaluations. Furthermore, due to the ever increasing amount of information available and changes to and development of treatment plans, health care providers accessing the TPES will essentially always be unaware of at least some of the included treatment plans and the efficacy of any particular treatment plan for a presenting patient.

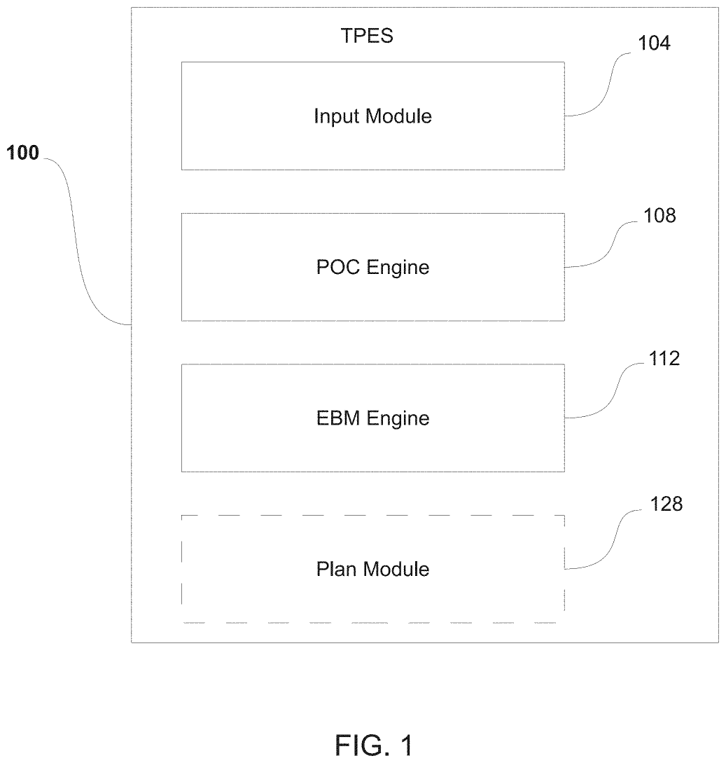

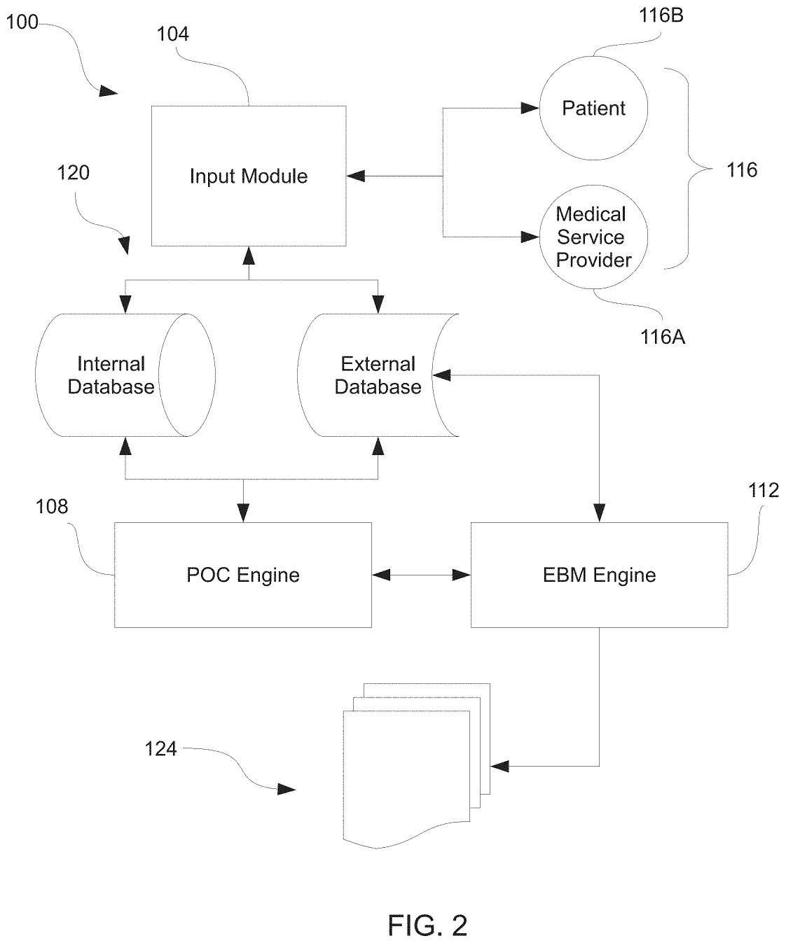

Turning now to the figures, and specifically with reference to FIGS. 1 and 2, at a high level, there is shown a TPES 100 according to an embodiment of the present disclosure. In this embodiment, TPES 100 includes an input module 104, a plan of care (POC) engine 108, and an evidence based medicine (EBM) engine 112.

Input module 104 is capable of receiving a plurality of inputs from different input sources 116, such as, but not limited to, one or more medical service providers 116A (e.g., doctor, nurse, therapist, office staff, billing coordinator, etc. and the like, which can be a disparately located facilities, and may be unaffiliated with one another) and a patient 116B. Medical service provider 116A can provide one or more diagnoses for a patient (e.g., primary diagnosis, secondary diagnosis, tertiary diagnosis, etc.) relating to the medical problem the patient is experiencing. In some embodiments, each diagnosis may be assigned a unique identification number by the system (“diagnosis codes”). Patient 116B can provide information regarding their symptoms, limitations (e.g., range of motion), pain intensity/severity, etc. In an exemplary embodiment, patient 116B supplies information related to a standardized outcome test (SOT), which is typically a series of questions or activities administered to patients and used to measure the existence and severity of a patient's functional deficits (e.g., the ability to walk, sleep, feed oneself, etc.). SOT data can be input directly by patient 116B using input module 104 (such as through a suitable graphical user interface (GUI)) or can be input by the medical service provider 116A or other representative. The SOT taken by patient 116B is typically chosen after diagnosis of the patient's medical condition by medical service provider 116A. For example, if medical service provider 116A determines that patient 116B is having a problem with their wrist, the medical service provider can decide among, for example, the DASH (disabilities of the aim, shoulder and hand) or the Mayo Wrist Score SOTs. Other SOTs are used in relation to different diagnoses, as understood by those in the medical service industry.

Input module 104 may be implemented as a web portal that may be used to facilitate access to information, patient care and/or practice management, for example. Information and/or functionality available via the web portal may include one or more of patient information, SOT questionnaires, clinical decision support, medication management, scheduling, medical resources, etc. In certain examples, the web portal serves as a central interface to access information and input information. For example, medical service provider 116A, after making a diagnosis of patient 116B, may be presented with a plurality of SOTs that relate to the diagnosis. Medical service provider 116A can chose one or more of the SOTs and the questions associated with the SOT may be viewed through the web portal or viewer by the patient 116B (at the same or a different physical location as the location of the medical service provider) or the medical service provider. Additionally, data entered into input module 104 may be manipulated and propagated using the web portal, and can be generated, modified, stored and/or used and then communicated to another application or system, such as POC engine 108, to be modified, stored and/or used.

In an exemplary embodiment, input module 104 may allow an input source 116 to track the performance and effectiveness of treatment plans based on, for example, improvements in SOT score values for a given diagnosis, or combination of diagnoses. This data may be stored in a database 120 which may be internal (i.e., only accessible at a certain location or by certain input sources 116) or may be external (i.e., stored so as to be accessible to all or a portion of TPES 100 input sources 116).

In an embodiment, input module 104 may be accessible locally (e.g., in an office) and/or remotely (e.g., via the Internet and/or other private network or connection). Input module 104 may be configured to help or guide a user in accessing data and/or functions to facilitate patient care and practice management, for example. In certain examples, input module 104 may be configured according to certain rules, preferences and/or functions, or a user may customize the input module (e.g., web portal) according to particular desires, preferences and/or requirements.

As used herein the term “plan of care”, “treatment plan”, or “POC” shall generally mean a medical treatment plan specific for one or more patient conditions or diagnoses chosen by a therapist or medical provider. In preferred embodiments, SOT data may be used to evaluate the effectiveness of a given treatment plan or POC. The system may assign a unique number or identifier to each plan of care (“POC ID”) or treatment plan (“treatment plan Number” or “Treatment Plan ID”).

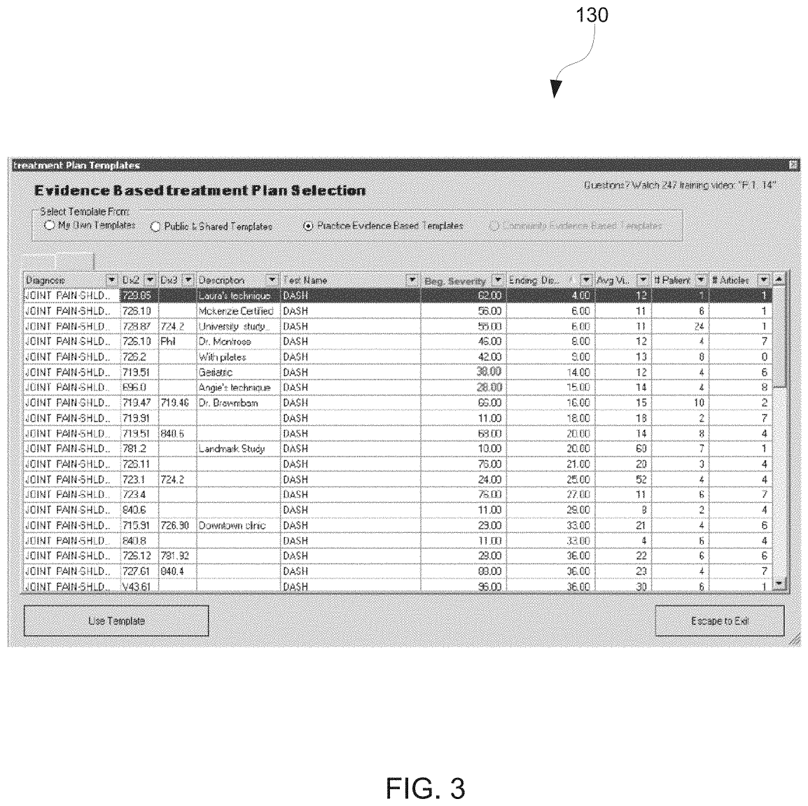

At a high level, POC engine 108 accepts information from input module 104 and prepares a selected list of treatment plans available to treat the patient. For example, POC engine 108 can first receive one or more diagnoses for a patient (e.g., primary, secondary, and tertiary diagnosis), with each diagnosis typically being assigned a unique identification number by the system (also referred to as “diagnosis codes”) (an example of such a query is shown in FIG. 3). POC engine 108 can also receive other information related to the diagnosis, such as, but not limited, to, diagnosis severity or functional deficits. For example, POC engine 108 can receive SOT data entered by patient 116A or a medical service provider employee using input module 104, where the SOT data is representative of the functional deficits of the patient. In an exemplary embodiment, POC engine 108 generates an SOT score based upon the SOT data. The term “SOT score” is typically a numerical value that can be computed by POC engine 108 corresponding to a patient's response to standard outcome tests or can assigned by input source 116. SOT scores are typically developed during the patient intake process at the initial evaluation, each re-evaluation, and each discharge visit. SOT scores are determined according to the SOT they relate to—in other words, for each SOT there is a methodology proposed for creating a SOT score for that specific test. Typically, the higher the SOT score the lower the impact on the patient. For example, patient 116B before undergoing treatment may enter SOT data in input module 104 that can result in an initial SOT score of 50 for an SOT related to the diagnosis of shoulder pain. After undergoing treatment plan Z, the patient may have an end SOT score of 75 for the same diagnosis.

In an exemplary embodiment, POC engine 108 can provide a means for an input source 116, such as medical service provider 116B, to select a database 120 to query (e.g., an internal database or external databases) based upon the information received by the POC engine. Upon receiving a selection from input source 116, POC engine 108 may query the appropriate database(s) to retrieve one or more treatment plans related to the information received, e.g., diagnosis, diagnosis code, SOT data, SOT score, diagnosis severity, etc. An example of such a query, query 130, is found in FIG. 3. In an exemplary embodiment, POC engine 108 is configured to selectively rank each treatment plan. The term “effective” as used herein shall generally mean plans of care which meets desired performance thresholds or other guidelines which may be specified by input source 116, payer, medical board, or other organization and which may change from time to time. In another exemplary embodiment, POC engine 108 may rank the treatment plans returned by the query based on, for example, % Avg Progress of SOT scores.

POC engine 108 can calculate a percent average progress (“% Avg. Progress”) value for a given treatment plan is as follows:

$$\% \text{Avg} \text{. Progress} = \frac{{\sum }_{i = 1}^{x} ( \frac{Δ SOTi}{Init SOTi} )}{x} * 100 \qquad Equation 1$$

a. Wherein:
i. ΔSOTᵢ=Score change between start and end/stoppage of treatment plan Init SOTᵢ=Initial SOT Score
ii. X=total number of patients who have undergone the treatment plan

In an exemplary embodiment, POC engine 108 can compare the average SOT initial value with the SOT score of patient 116B for all plans meeting the diagnosed condition. In this embodiment, POC engine can return a list of treatment plans to medical treatment provider 116A that are those plans meeting the diagnosed condition which also have an SOT score comparable to what patient 116A's SOT score is. As used herein, “comparable to” means a value that is within a certain amount or percentage, which may vary as a function of the number of treatment plans available for the diagnosed condition. For example, if only a few treatment plans are available for certain diagnosis, all treatments plans would be shown. In another example, where many treatment plans are available, POC engine 108 may provide a list of those treatments plans within about 10% of patient 116B's SOT score. In an exemplary embodiment, the list may be displayed according to one or more outcome measures, e.g., number of visits, % Avg Progress, SOT final score, etc., by diagnosis type and SOT.

POC engine 108 can also compare and filter treatment plans based on other desired outcome criteria, such as, but not limited to, an average number of visits, a % Avg. Progress, an average final SOT score, and a diagnosis severity. In an exemplary embodiment, POC engine 108 can communicate with a database 120 that contains a digital collection of data or information such as, but not limited to, patient profile data, SOT data, SOT scores, treatment plan information, outcome criteria, and citations and links for research studies, journal articles, and the like (also referred to herein as supporting information or documentation). Database 120 can be stored on a remote server and accessed by input source 116 (via, for example, a computer) through the internet or may be stored on computer accessible by a local area network.

POC engine 108 can also develop an average rate of improvement for each treatment plan for each diagnosis category (as well as, optionally, each level of diagnosis severity) by evaluating the initial SOT score, the final SOT score, and the duration of treatment for each patient who underwent that treatment plan. This average rate of improvement can be used to select a treatment plan for a patient based on the treatment plan(s) with the highest rate of improvement, which can be considered the likely most effective treatment plan for the patient. In addition, the determined rate of improvement can be updated based on the results of newly implemented treatment plans. As additional patients undergo a treatment plan or additional information is determined for patients undergoing a treatment plan, information is added to the database and each pertinent value is updated. In this way, information is collected and data is updated in real-time such that disparately located health care providers have access to the most recent information about treatment plans (as well as new treatment plans they could not otherwise be aware of) that may be associated with a diagnosis for a current patient. Moreover, the most likely effective treatment plan is selected objectively and not skewed by the necessarily limited experiences of an individual health care provider.

EBM engine 112 communicates with input module 104 and POC engine 108 and generates electronic documentation to be used in defense of treatment plans which may be suggested to a user 116 or TPES 100. In an exemplary embodiment, EBM engine 112 is configured to generate documentation and supporting information 124 for each treatment chosen by TPES' 100 or input source 116 along with corresponding patient information, patient diagnoses, billing codes, and evidence based data in support of each treatment plan. EBM engine 112 may be configured to send supporting documentation for appending to a patient's electronic medical record (EMR) or export the documentation a payer's claim system for reimbursement. In an exemplary embodiment, EBM engine 112 is configured to properly format claims and generate appropriate billing codes for a plurality of different payer claim systems and billing requirements.

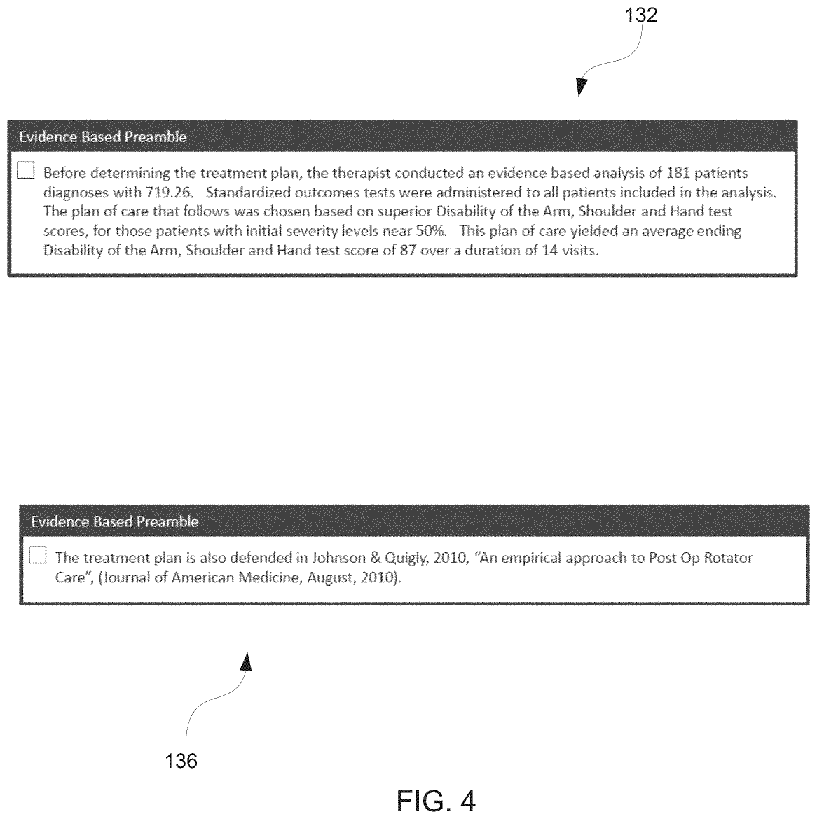

For example, EBM engine can produce a statement such as that found in FIG. 4. As shown, the statement includes two primary aspects, supporting information 132 and evidence statement 136. Supporting information 132 can include information related to the plan implemented and the statistical history of that plan. For example, and as found in FIG. 4, supporting information 132 includes a statement of the diagnosis, a statement regarding the average improvement, an ending SOT score, and a number of visits. Evidence statement 136 can include information related to journals, articles, etc., that support the chosen plan. The materials in evidence statement 136 can be uploaded by any medical service provider 116A or other parties involved in the creation or evaluation of treatment plans.

In another exemplary embodiment of TPES 100, the TPES can include a plan module 128, which allows for the creation, retention, and future use of a modified or new treatment plan that is not currently available in database 120 for a given diagnosis or diagnosis/SOT score combination. In an exemplary embodiment, when material changes, such as, but not limited to, an addition of a specific exercise or a therapy; the elimination of a portion or feature of a treatment plan: or the substitution of an element of a treatment plan for a different element, plan module 128 tracks the change. Non-material changes, include, but are not limited to, changes to the number of repetitions of an exercise, sets of an exercise, amount of resistance or weight used during the exercise, within an existing treatment plan. Once a material change is made, plan module 128 determines whether an existing plan in database 120 exists that includes the revised treatment plan. If yes, plan module 128 associates the patient's treatment with the existing plan. If no, plan module 128 creates a new treatment plan in database 120 consisting of the prior treatment plan and any modifications to that prior plan. The new plan created by plan module 128 can then be updated as outcomes become available, is usable by other medical service providers 116A, and supporting documentation can be associated with the new plan (note that supporting documentation associated with the prior plan does not necessarily automatically carry over as the plan has changed). As a non-limiting illustrative example: a medical service provider 116A uses TPES 100 to select a treatment plan titled “Cervical 3D” from database 120 based upon patient 116B's functional deficits and diagnosis. Cervical 3D consists of treatment options 97035, Ultrasound; 97112, Neuro Re-ed; and 97110, Therex. After six visits with medical service provide 116A, the medical service provider adds treatment option 97140, Manual Therapy, to the treatment plan. Upon addition of this material change, plan module 128 recognizes that the prior plan, “Cervical 3D”, is no longer being used and stores the new, updated treatment plan, and then tracks the outcomes separately from the Cervical 3D plan. In this way, treatment progression can be tracked for each patient and each different treatment plan, which allows for statistical analysis of which treatment plans are superior at restoring the identified functional deficit for a given diagnosis.

Turning now to FIG. 4, there is shown an exemplary method 200 for developing an evidenced based treatment plan for a patient having functional deficit.

At step 204, the patient, such as patient 116B is diagnosed with a medical condition by a medical service provider, such as medical service provider 116A. In an exemplary embodiment, the diagnosis is associated with a category or identification number. Additional diagnoses or subcategories of diagnosis can also be categorized by the medical service provider. For example, if the patient has a shoulder condition, a first level or primary diagnosis may be shoulder diagnosis having a classification A001, and a secondary diagnosis may be a rotator cuff strain having a classification B01. The number and degree of classification available to the medical service provider is often based on the type of diagnosis experienced by the patient. The diagnosis information can be input into an input module, such as input module 104.

At step 208, SOT data are input in response to an SOT given to the patient, such as patient 116B. As described above with reference to FIG. 1, SOT data can be input directly by the patient in respond to a series of questions available, for example, on a computer screen or a medical service provider can enter the responses of the patient to the questions. The type of SOT is typically chosen by the medical service provider and corresponds to the diagnosis.

At step 212, an SOT score is generated. As discussed above, each SOT has its own algorithmic formula for computation of a score associated with the SOT input data. Thus, at step 212, the SOT input data from step 208 are input into its respective algorithm for generation of the SOT score.

At step 216, a database containing information related to treatment plans, such as, but not limited to, the details of the treatment plans (e.g., exercises, stretches, tests, etc.), the outcomes related to those treatment plans, and supporting documentation for the treatment plans is accessed. The information gathered in steps 204, 208, and 212 can also be added to the database so as to create an additional patient record that can serve (when outcomes are input after completion of the treatment plan) to inform future medical service providers about the applicability of the treatment plan for a patient having a similar condition.

At step 220, a list of treatment plans is determined. The list may be determined by evaluating or comparing the diagnosis of the patient with the treatment plans applicable to the diagnosis. In another exemplary embodiment, the list may be further refined by comparing the average starting SOT score of patients who have used a given treatment plan with the SOT score of the patient. In yet another exemplary embodiment, the list may be further refined by comparing the average diagnosis severity of patients who have used a given treatment plan with the diagnosis severity experienced by the current patient. In yet a further embodiment, the list may be further refined by querying the database for certain outcome measures, such as but not limited to, average number of visits, average change in SOT score, final SOT score, etc. An example of treatment plans for a given diagnosis are found in FIG. 3.

At step 224, a treatment plan is selected. The treatment plan chosen for the patient can be based upon predefined criteria, such as, but not limited to, best overall outcome (e.g., lowest SOT score, largest change in average SOT score, etc.), existence of supporting documentation, etc.

At step 228, an evidence based document (EBD) statement is prepared. The EBD statement is based upon supporting information contained in a database that is related to the treatment plan. An example of such a statement is found in FIG. 4.

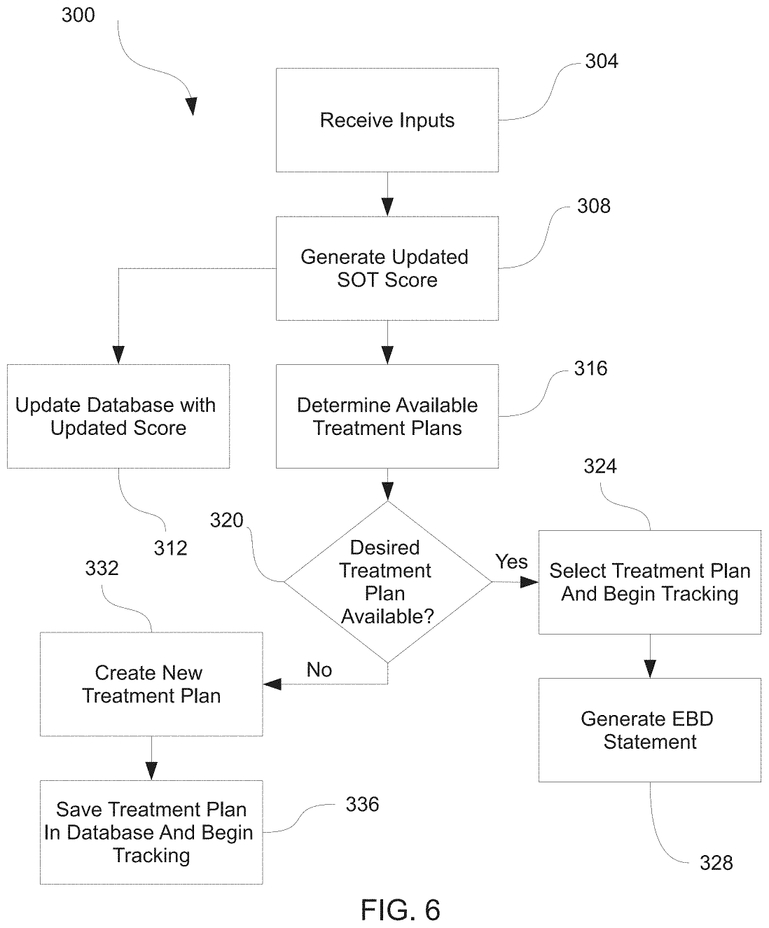

In the event that a medical service provider, such as medical service provider 116A, is dissatisfied with the previously chosen treatment plans, desires to reevaluate/modify the treatment plan, desires an alternative treatment plan, or, the patient having completed the prior plan, needs a new plan, based upon the functional deficits or diagnosis severity being experienced by the patient, the medical service provider can use process 300, shown in FIG. 6, to include, select, or develop a new treatment plan in the database.

At step 304, process 300 receives inputs, such as, but not limited to, updated diagnoses, updated SOT data, and updated diagnosis severity. In an exemplary embodiment, the diagnosis is associated with a category or identification number. Additional diagnoses or subcategories of diagnosis can also be categorized by the medical service provider. For example, if the patient has a shoulder condition, a first level or primary diagnosis may be shoulder diagnosis having a classification A001, and a secondary diagnosis may be a rotator cuff strain having a classification B01. The number and degree of classification available to the medical service provider is often based on the type of diagnosis experienced by the patient. The diagnosis information can be input into an input module, such as input module 104. SOT data are input in response to an SOT given to the patient, such as patient 116B. As described above with reference to FIG. 1, SOT data can be input directly by the patient in respond to a series of questions available, for example, on a computer screen or a medical service provider can enter the responses of the patient to the questions. The type of SOT is typically chosen by the medical service provider and corresponds to the diagnosis.

At step 308, an updated SOT score is generated (in a similar fashion to the generation found in step 212 of process 200). Depending on the situation, this may be a final SOT score (post-treatment) or a mid-treatment SOT score. In certain embodiments of a TPES 100 and the processes described herein, the TPES can continually track the progress of the patient and thus, SOT scores are updated on each visit with the patient's medical service provider.

At step 312, a database containing information related, to treatment plans and outcomes, among other information, receives the updated SOT score and other information received by process 300. This information can be used, along with other information, to improve the knowledge store for future users of the treatment plan.

At step 316, the database containing information related to treatment plans, such as, but not limited to, the details of the treatment plans (e.g., exercises, stretches, etc.), the outcomes related to those treatment plans, and supporting documentation for the treatment plans is accessed. The available plans may be determined by evaluating or comparing the diagnosis of the patient with the treatment plans applicable to the diagnosis. In another exemplary embodiment, the list may be further refined by comparing the average starting SOT score of patients who have used a given treatment plan with the SOT score of the patient. In yet another exemplary embodiment, the list may be further refined by comparing the average diagnosis severity of patients who have used a given treatment plan with the diagnosis severity experienced by the current patient. In yet a further embodiment, the list may be further refined by querying the database for certain outcome measures, such as but not limited to, average number of visits, average change in SOT score, final SOT score, a rate of improvement based on initial and final SOT scores and duration of treatment, etc.

At step 320, a determination is made as to whether the desired treatment plan is available. Desirability can be determined from predetermined criteria, such as, but not limited to, the existence of a specific treatment option, a duration, etc.

If a plan is found, at step 324, a treatment plan is selected. The treatment plan chosen for the patient can be based upon predefined criteria, such as, but not limited to, best overall outcome (e.g., lowest SOT score, largest change in average SOT score, etc.), existence of supporting documentation, etc.

At step 328, an evidence based document (EBD) statement is prepared for the selected plan. The EBD statement is based upon supporting information contained in a database that is related to the treatment plan. An example of such a statement is found in FIG. 4.

If no appropriate plan is available for the patient, process 300 turns to step 332, where a new plan is created. The new plan can be based on an existing plan or can be an entirely new treatment plan. Step 332 creates the standard fields for setup of the plan, e.g., treatment therapies, and relates the plan to given diagnosis for future retrieval.

At step 336, the new plan is saved in the database and future events involving the plan are tracked. The new plan is available for other medical service providers serving other patients.

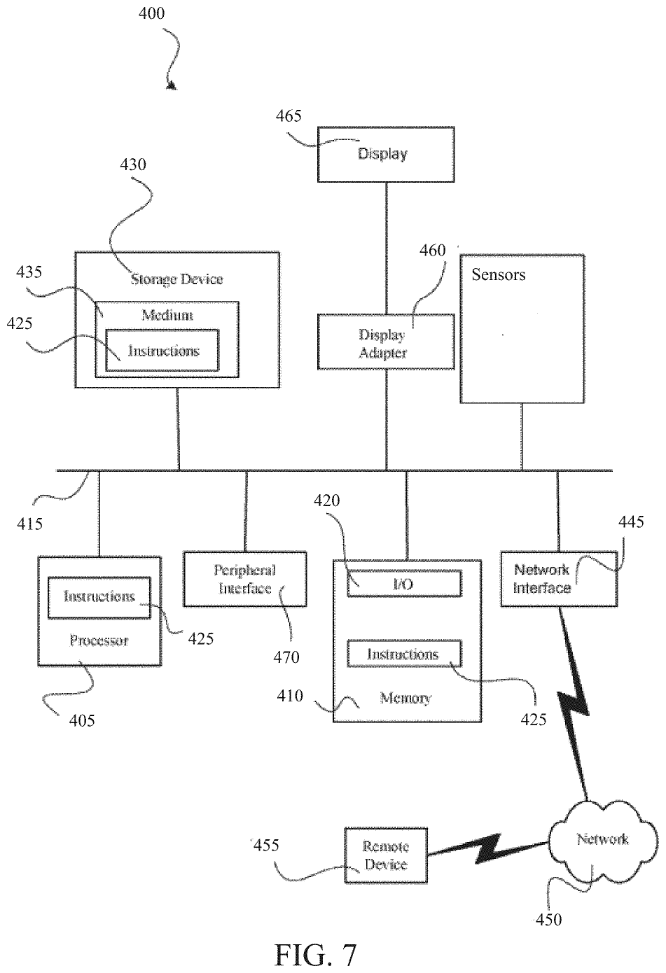

FIG. 7 shows a diagrammatic representation of one implementation of a machine/computing device 400 that can be used to implement a set of instructions for causing TPES 100 to perform any one or more of the aspects and/or methodologies of the present disclosure. Device 400 includes a processor 405 and a memory 410 that communicate with each other, and with other components, such as database 120, via a bus 415. Bus 415 may include any of several types of communication structures including, but not limited to, a memory bus, a memory controller, a peripheral bus, a local bus, and any combinations thereof, using any of a variety of architectures.

Memory 410 may include various components (e.g., machine-readable media) including, but not limited to, a random access memory component (e.g., a static RAM “SRAM”, a dynamic RAM “DRAM”, etc.), a read-only component, and, any combinations thereof. In one example, a basic input/output system 420 (BIOS), including basic routines that help to transfer information between elements within device 400, such as during start-up, may be stored in memory 410. Memory 410 may also include (e.g., stored on one or more machine-readable media) instructions (e.g., software) 425 embodying any one or more of the aspects and/or methodologies of the present disclosure. In another example, memory 410 may further include any number of program modules including, but not limited to, an operating system, one or more application programs, other program modules, program data, and any combinations thereof.

Device 400 may also include a storage device 430. Examples of a storage device (e.g., storage device 430) include, but are not limited to, a hard disk drive for reading from and/or writing to a hard disk, a magnetic disk drive for reading from and/or writing to a removable magnetic disk, an optical disk drive for reading from and/or writing to an optical media (e.g., a CD, a DVD, etc.), a solid-state memory device, and any combinations thereof. Storage device 430 may be connected to bus 415 by an appropriate interface (not shown). Example interfaces include, but are not limited to, SCSI, advanced technology attachment (ATA), serial ATA, universal serial bus (USB), IEEE 1395 (FIREWIRE), and any combinations thereof. In one example, storage device 430 may be removably interfaced with device 400 (e.g., via an external port connector (not shown)). Particularly, storage device 430 and an associated machine-readable medium 435 may provide nonvolatile and/or volatile storage of machine-readable instructions, data structures, program modules, and/or other data for TPES 100. In one example, instructions 425 may reside, completely or partially, within machine-readable medium 435. In another example, instructions 425 may reside, completely or partially, within processor 405.

Device 400 may also include a connection to one or more sensors. Sensors may be interfaced to bus 415 via any of a variety of interfaces (not shown) including, but not limited to, a serial interface, a parallel interface, a game port, a USB interface, a FIREWIRE interface, a direct connection to bus 415, and any combinations thereof. Alternatively, in one example, a user of device 400 may enter commands and/or other information into device 400 via an input device (not shown). Examples of an input device include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), touchscreen, and any combinations thereof.

A user may also input commands and/or other information to device 400 via storage device 430 (e.g., a removable disk drive, a flash drive, etc.) and/or a network interface device 445. A network interface device, such as network interface device 445, may be utilized for connecting device 400 to one or more of a variety of networks, such as network 450, and one or more remote devices 455 connected thereto. Examples of a network interface device include, but are not limited to, a network interface card, a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a direct connection between two computing devices, and any combinations thereof. A network, such as network 450, may employ a wired and/or a wireless mode of communication. In general, any network topology may be used. Information (e.g., data, instructions 425, etc.) may be communicated to and/or from device 400 via network interface device 455.

Device 400 may further include a video display adapter 460 for communicating a displayable image to a display device 465. Examples of a display device 465 include, but are not limited to, a liquid crystal display (LCD), a cathode ray tube (CRT), a plasma display, and any combinations thereof.

In addition to display device 465, device 400 may include a connection to one or more other peripheral output devices including, but not limited to, an audio speaker, a printer, and any combinations thereof. Peripheral output devices may be connected to bus 415 via a peripheral interface 470. Examples of a peripheral interface include, but are not limited to, a serial port, a USB connection, a FIREWIRE connection, a parallel connection, a wireless connection, and any combinations thereof.

A digitizer (not shown) and an accompanying pen/stylus, if needed, may be included in order to digitally capture freehand input. A pen digitizer may be separately configured or coextensive with a display area of display device 465. Accordingly, a digitizer may be integrated with display device 465, or may exist as a separate device overlaying or otherwise appended to display device 465.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for determining a likely most effective treatment plan for a patient in need thereof, the patient having identified and quantified functional deficits and a patient diagnosis category determined by a health care provider, the system comprising:
a database including information related to a plurality of other patients, the information including at least one diagnosis category, an initial standardized outcome test (SOT) score, a final SOT score, a treatment plan, and a treatment plan duration, wherein at least one of the associated treatment plans is unknown to the health care provider or wherein the health care provider is unaware as to an efficacy of any particular treatment plan; and
a computing device including a processor and a non-transitory computer readable medium in communication with the processor and the database, wherein said non-transitory computer readable medium includes instruction that cause the processor to:
generate a SOT score based upon the patient's functional deficits;
identify a plurality of the treatment plans that address the patient diagnosis category;
compare the SOT score to the initial SOT scores of the plurality of treatment plans; and
select, from the plurality of identified treatment plans, the likely most effective treatment plan by evaluating the final SOT score and the treatment plan duration so as to develop a rate of improvement for each of the plurality of identified treatment plans, wherein the treatment plan with the highest rate of improvement is the likely most effective treatment plan.

2. A method for selecting a treatment plan for patients having identifiable functional deficits, the method comprising:
providing a database accessible to a plurality of disparately located health care providers, each of the health care providers having a plurality of patients;
receiving information from the plurality of disparately located health care providers, the information including initial standardized outcome test (SOT) data, final SOT data, diagnosis category, treatment plan duration, and treatment plans, wherein each of the forgoing relating to each of the plurality of patients;
evaluating the information so as to develop a rate of improvement for each diagnosis category based upon the treatment plan duration, the initial SOT data and the final SOT data; and
selecting a likely most effective treatment plan based upon the treatment plan with the highest rate of improvement.

3. A method of improving patient treatment plans comprising:
providing a database accessible to a plurality of disparately located health care providers, each of the health care providers having a plurality of patients;
receiving information from the plurality of disparately located health care providers, the information including initial standardized outcome test (SOT) data, final SOT data, diagnosis category, treatment plan duration, and treatment plans, wherein each of the forgoing relating to each of the plurality of patients;
evaluating the information so as to develop a rate of improvement for each diagnosis category based upon the treatment plan duration, the initial SOT data, and the final SOT data; and
updating the rate of improvement based upon additional information received from the plurality of disparately located health care providers.
